(19) United States
(12) Patent Application Publication
(10) Pub. No.: US 2025/0161584 A1
(43) Pub. Date: May 22, 2025

(54) PRECISION DISPENSING

(71) Applicant: Innomed Technologies, Inc., Encino, CA (US)

(72) Inventors: John H. JOSEPH, Westlake, CA (US); Michael A. BOTTA, Ridge, NY (US); Daniel J. DUMINUCO, Holtsville, NY (US); Jake A. TRAUB, Encino, CA (US)

(21) Appl. No.: 18/912,446

(22) Filed: Oct. 10, 2024

Related U.S. Application Data
(60) Provisional application No. 63/543,723, filed on Oct. 11, 2023.

(51) Int. Cl. A61M 5/315

(52) U.S. Cl. CPC A61M 5/3158; A61M 5/31566; A61M 2205/0216; A61M 2205/583

(57) ABSTRACT

A syringe carriage is adapted to engage a syringe plunger and to slide relative to the syringe cartridge by user motion applied to a roller element that frictionally engages and rolls on a slide surface of the syringe cartridge, thereby facilitating precise movement of the syringe plunger relative to the syringe cartridge and thus precision dispensing of the syringe contents. The syringe cartridge may include a viewport a spine surface generally opposite the viewport and having indicia thereon. The spine surface, syringe lumen and viewport may provide for magnification of the indicia, through the syringe contents and viewport to the user. The syringe carriage may be releasably secured to the syringe cartridge using a flexible fastening assembly on the syringe cartridge, such that syringe cartridges may be installed and removed quickly and easily on the syringe carriage such that the syringe carriage may be used with multiple syringe cartridges and thus form kits.

### RELATED APPLICATIONS AND PRIORITY CLAIM

Priority is claimed to U.S. Provisional Application No. 63/543,723 titled PRECISION DISPENSING and filed on Oct. 11, 2023. The subject matter and disclosure of the aforementioned application is incorporated herein by reference in its entirety.

### FIELD

The disclosure relates generally to dispensing devices and medical devices, such as syringes for controlling delivery of contents, such as medication and therapeutic fluids, to a patient. The disclosure also relates to features for enhancing visual indicators of syringe components and syringe contents, such as indicators of the nature (i.e., identity of the contents or contents manufacturer) and volume of such contents. The disclosure relates further to kits comprising a syringe attachment or carriage and multiple syringe cartridges that may each be releasably attached to and used with the attachment or carriage. The disclosure further relates to syringe features that are adapted to provide precise and ergonomically comfortable control of dispensing of contents from, and operation of, syringes and dispensing devices.

### BACKGROUND

A number of problems in the prior art stem from the limitations of existing syringes to be adapted to precision, very low volume applications, such as delivery of very small amounts of botulinum toxin. For example, the ability to deliver precise volumes in increments smaller than 0.01 ml may typically be advantageous. Existing syringe configurations are not readily adapted to precise control of delivery such small increments. Existing syringe configurations are similarly not ergonomically adapted to permit precise control of dispensing by a user. In addition, a related problem is accurate control and reading of visual indicators on syringes as the syringe form factor becomes reduced. Such visual indicators may include the volume of syringe contents in the syringe lumen, the position of the syringe plunger and/or plunger piston, and the condition (i.e., whether bubbles are present) and nature or identify of the syringe contents. Ergonomic factors, including user comfort and control and readability of syringe indicia and plunger position also become a factor as the lumen diameter and other parameters are reduced.

It would therefore be advantageous to provide devices, including syringes and attachments that address the aforementioned shortcoming and others in the prior art.

### SUMMARY

According to an aspect of the disclosure, a syringe carriage is adapted to engage a syringe plunger and to slide relative to the syringe cartridge by user motion applied to a roller element that frictionally engages and rolls on a slide surface of the syringe cartridge, thereby facilitating precise movement of the syringe plunger relative to the syringe cartridge and thus precision dispensing of the syringe contents. According to a further aspect of the disclosure, the syringe cartridge may include a viewport for allowing a user to view contents (liquid) in the syringe lumen and a spine surface generally opposite the viewport and having indicia thereon. According to an aspect, dimensions and geometry of the spine surface, syringe lumen and viewport may provide for magnification of the indicia thereon to the user. More particularly, the lumen, when filled with transparent or translucent syringe contents, may function as a cylindrical (circular cross-section) lens which magnifies the indicia on the spine surface when a user views the indicia in the viewport (and through the cylindrical lens formed by the syringe contents in the lumen). In this manner, indicia can be magnified and further, syringe contents can be made to appear to be a certain hue or color. For example, an axially extending line or strip of a particular color (blue, pink or green) may be provided on the spine surface such that the syringe contents appear to the user, when viewing the syringe contents through the viewport, to take on the color of the axially extending line or strip. Syringe contents, even though clear or colorless may be made to appear to the user to have the particular color. Thus, aspects of the disclosure provide for the development or use of color-based branding for manufacturers of syringe contents without requiring the syringe contents to be modified (i.e., may be pigmented or colored). Plastic material selected for the syringe cartridge may have suitable optical properties (i.e., transparency or translucency) such that the spine surface and indicia thereon may be readily viewed by the user from the opposite side of the syringe cartridge (i.e., within the viewport).

According to some embodiments and aspects of the disclosure, a syringe cartridge may comprise a main body including a lumen defined on an interior thereof and a viewport defined in main body and arranged to permit a user to view syringe contents in the lumen. In a further aspect, the syringe cartridge may include a spine extending axially along the main body and having a spine surface, wherein the spine surface is arranged such that indicia on the spine surface appear magnified to a user when viewed through contents in the syringe lumen and the viewport. In a further aspect, the syringe cartridge may comprise a fastening assembly on the main body, the fastening assembly configured to secure a carriage to the cartridge. In some aspects, the may comprise a plunger and a carriage adapted to engage an end of the plunger, the carriage configured to slide relative to the main body. In some aspects, the syringe carriage may comprise a roller on the carriage adapted to frictionally engage the syringe cartridge. In some aspects, the syringe carriage may comprise a flexible mount on the carriage for mounting the roller and allowing it to be depressed by a user to frictionally engage the cartridge surface to allow plunger position to be controlled by rolling movement of the roller.

According to some further aspects, the main body may have a wall thickness surrounding the lumen and the viewport may extend to a depth such that the syringe contents are viewed through a thickness of material that is less than or equal to the wall thickness surrounding the lumen. In some further aspects, the syringe cartridge may a lateral viewing area defined on the main body be adapted to permit a user to view the syringe contents from a side of the syringe body. In some aspects, the viewport may be an axially extending trough or slot formed in the syringe cartridge to a depth that provides a thin wall to view the lumen contents. In some aspects, the trough may extends to a depth in the main body such that syringe contents are clearly visible to a user. In some aspects, the fastening assembly may comprise a guide surface on the syringe cartridge main body for slidably engaging a carriage and at least one releasable fastener for securing the carriage to the syringe cartridge.

According to an aspect of the disclosure, a syringe carriage is adapted to engage a syringe plunger and to slide relative to the syringe cartridge by user motion applied to a roller element that frictionally engages and rolls on a slide surface of the syringe cartridge, thereby facilitating precise movement of the syringe plunger relative to the syringe cartridge and thus precision dispensing of the syringe contents. According to an aspect of the disclosure, the syringe carriage may be releasably secured to the syringe cartridge with a fastening assembly on the syringe cartridge such that it may be installed and removed quickly and easily such that the syringe carriage may be used with multiple syringe cartridges provided in kits of a single carriage with multiple cartridges.

According to some aspects and embodiments, a syringe cartridge may comprise a main body including a lumen defined on an interior thereof, a slide extending from the main body and including a generally planar surface and a carriage fastening assembly disposed on the main body. The carriage fastening assembly may be configured to releasably and slidably secure a carriage to the syringe cartridge. According to some further aspects, the syringe cartridge may comprise a syringe carriage adapted to engage a syringe plunger and adapted to slide relative to the syringe cartridge to move the plunger relative thereto, the syringe carriage having at least one rail defined thereon, wherein the fastening assembly is configured to engage and retain the at least one rail. According to some further aspects, the syringe cartridge may further comprise a syringe carriage adapted to engage a syringe plunger and adapted to slide relative to the syringe cartridge to move the plunger relative thereto, wherein the carriage fastening assembly includes a pair of pivoting levers configured to capture and secure a carriage for sliding movement relative to the syringe cartridge.

According to further aspects, the syringe cartridge may comprise a syringe carriage adapted to engage a syringe plunger and adapted to slide relative to the syringe cartridge to move the plunger relative thereto, wherein the syringe carriage includes a pair of projections configured to secure the carriage to the slide with a snap fit and to guide the carriage for sliding movement on the slide. According to further aspects, the syringe cartridge may further comprise a syringe carriage adapted to engage a syringe plunger and adapted to slide relative to the syringe cartridge to move the plunger relative thereto, wherein the syringe carriage includes a roller configured to frictionally engage the slide and allow a user to precisely move the carriage relative to the syringe cartridge. According to some further aspects, the fastening assembly may comprise a pair of lower guide surfaces arranged to slidingly engage a lower surface of the carriage, and pair of upper guide surface arranged to slidingly engage an upper surface of the carriage such that the carriage is captured for sliding movement between the lower guide surfaces and upper guide surfaces.

### DESCRIPTION OF THE DRAWINGS

The attendant advantages and features of the disclosure will be apparent from the following detailed description together with the accompanying drawings, in which like reference numerals represent like elements throughout. It will be understood that the description and embodiments are intended as illustrative examples according to aspects of the disclosure and are not intended to be limiting to the scope of invention, which is set forth in the claims appended hereto.

FIG. 1 is an isometric view of an example syringe assembly including a syringe cartridge and syringe carriage, according to aspects of the disclosure.

FIG. 2 is an exploded view of the syringe assembly of FIG. 1.

FIG. 3 is a bottom view of the syringe cartridge of FIG. 1.

FIG. 4. is a cross-section of the syringe cartridge of FIG. 3 in the plane 4-4 in FIG. 3.

FIG. 5 is a top view of the syringe cartridge of FIG. 3.

FIG. 6 is a cross-section of the syringe cartridge of FIG. 3 in the plane 6-6 of FIG. 3.

FIG. 7 is rear view showing details of a fastening assembly of the syringe cartridge of FIG. 3.

FIG. 8 is a bottom view of a syringe attachment or carriage according to aspects of the disclosure.

FIG. 9 is a side view of the carriage of FIG. 8.

FIG. 10 is a cross-section in the plane 10-10 in FIG. 8.

FIG. 11 is an enlarged view of the details in view 11 in FIG. 8.

FIG. 12 is a front view (from the dispensing end) of the carriage of FIG. 8.

FIG. 13 is an isometric view of the carriage of FIG. 8.

FIG. 14 is an isometric view of the syringe assembly showing the carriage in an installation position.

FIG. 15 is an isometric view of the syringe assembly showing the carriage in an installed position.

FIG. 16 is a side view of the syringe cartridge of FIG. 3.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein comport with standards used by those experienced or skilled in the art to most effectively convey the substance of their work to others skilled in the art. In some instances, well-known methods, procedures, and components have not been described in detail to avoid unnecessarily obscuring aspects of the disclosure.

References in the specification to “aspects of the disclosure” or to “some embodiments” indicate that the embodiment described may include a particular feature, structure, or characteristic, but every embodiment may not necessarily need to include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to apply such feature, structure, or characteristic in connection with other embodiments whether or not explicitly described.

The following examples are illustrative, but not limiting, of the present disclosure. Other suitable modifications and adaptations of the variety of conditions and parameters normally encountered in the field, and which would be apparent to those skilled in the art, are within the spirit and scope of the disclosure.

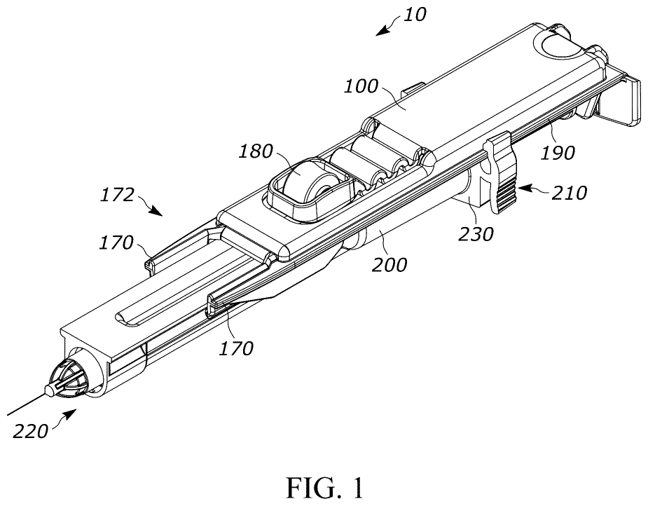

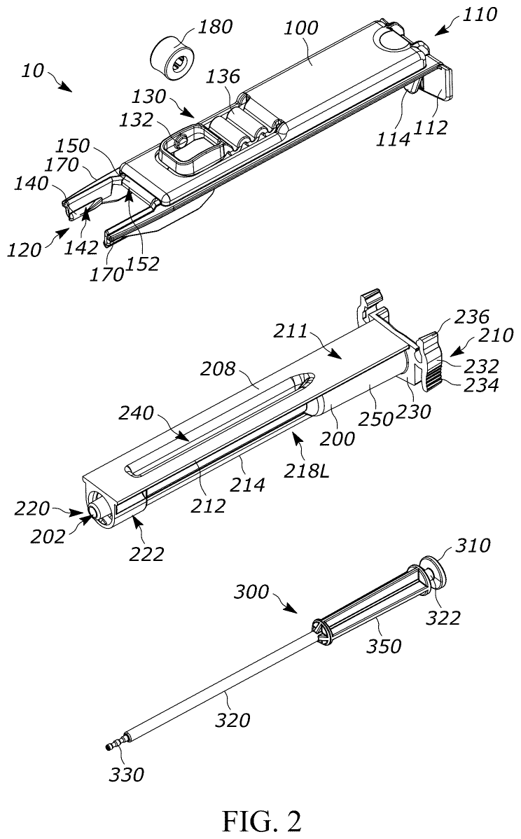

FIGS. 1 and 2 show an assembled view and an exploded view, respectively, of an example syringe assembly 10, including a syringe carriage or attachment 100 and a syringe cartridge 200, both according to aspects of the disclosure. Carriage 100 is adapted to be releasably engaged to and to be guided and move axially in a very precise manner relative to the syringe cartridge 200 when carriage roller 180 is operated by a user's thumb, as will be further detailed herein. Syringe carriage 100 may be reused interchangeably with different syringe cartridges 200, which may be standardized, single-use and prefilled. As will be recognized, and in accordance with aspects of the disclosure, these components facilitate packaging and sale in kits carriage/syringe cartridge kits that may include multiple (i.e., ten or more) single-use syringe cartridges 200. These aspects of the syringe cartridges 200 provide manufacturing and quality control advantages. For example, the syringe cartridges 200 may be made, pre-filled and processed en masse in a controlled environment, which may provide for sanitization and tamper-proof sealing/packaging of each of the syringe cartridges 200 and later used in the field with the carriage 100.

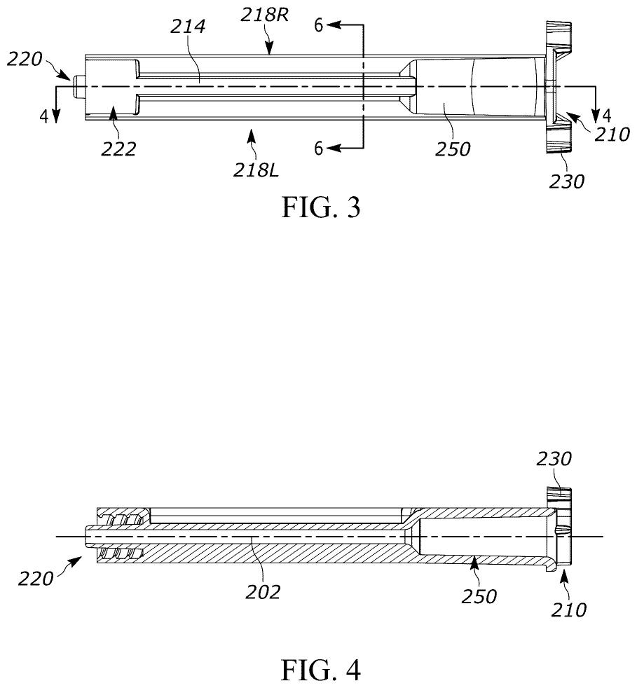

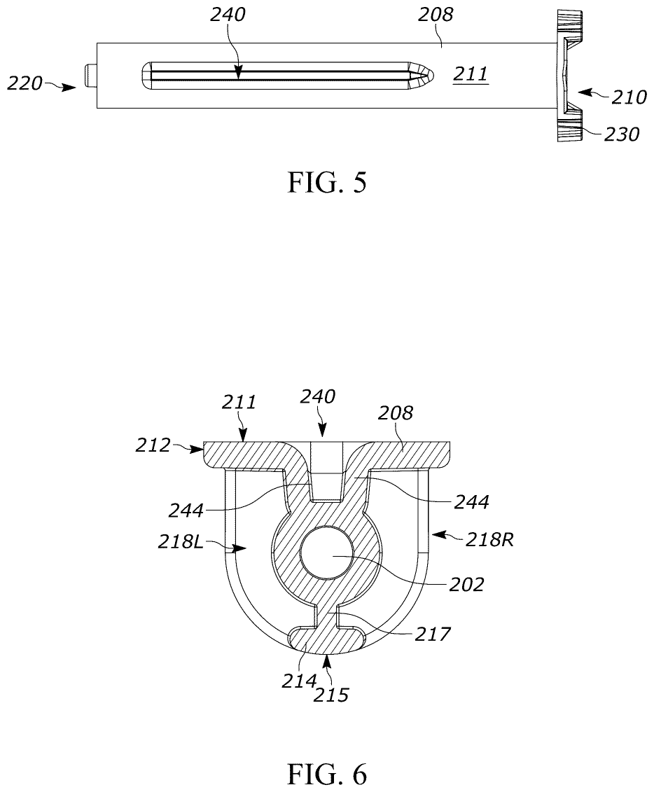

Referring additionally to FIGS. 3-7, syringe cartridge 200 includes a main body defining an internal, generally cylindrical lumen 202 extending axially from a plunger end 210 to a dispensing end 220 of the syringe cartridge 200. According to aspects of the disclosure, plunger end 210 may include a fastening assembly 230 for releasably securing the carriage 100 to the syringe cartridge 200 as will be further described. An axially extending slide 208, which may be a web or flange formed as part of the syringe cartridge main body provides an upper, generally planar surface 211 and two opposed side surfaces 212 upon which the carriage 100 may slide and be guided as it moves in an axial direction relative to the syringe cartridge 200. Fastening assembly 230 includes a pair of pivoting fastening levers 232 with actuating ends 234 and fastening ends 236. When a user squeezes the actuating ends 234 together, the fastening ends 236 pivot away from each other and permit the installation or removal of the carriage 100 from the syringe cartridge 200 as will be further detailed herein. Dispensing end 210 includes a threaded connector for receiving a needle hub and sealing the needle hub to the syringe cartridge 200 in such a manner that the needle lumen is in fluid communication with the syringe cartridge lumen.

According to aspects of the disclosure, syringe cartridge 200 includes an axially extending spine 214 along a bottom portion thereof. Spine 214 may be supported on an orthogonal rib 217 (extending radially from the syringe cartridge barrel) and provides a gripping surface for a user's fingers and for indicia. According to aspects of the disclosure, syringe 200 may include features for enhanced viewing and visibility of the lumen interior and syringe contents (such as medical or therapeutic fluids as well as the syringe plunger and piston) occupying the lumen. This allows enhanced viewing of the position of syringe plunger 300 and plunger piston 310, and the position (volume) of the syringe contents within the lumen. An elongate viewport 240 may comprise a trough formed in the top of the slide 208 and extending generally axially and parallel to the syringe lumen 202.

Referring particularly to FIG. 6, the viewport 240 may extend to a depth such that the wall separating the viewport and the lumen 202 is sufficiently thin to permit clear viewing, from the top of the syringe cartridge 200, of the syringe contents contained in the cylindrical lumen 202 as well as the position of the syringe plunger and/or plunger piston. For further enhanced viewing or perception of syringe contents, viewport 240 may include lateral sidewalls 244 extending upward from the lumen wall to the upper surface of slide 208 at a slight outward angle. Lumen 202, when occupied with clear or translucent contents, may operate as a cylindrical lens with magnification properties. Indicia (i.e., gradations or markings) or coloration (i.e., a colored axially extending line) may be provided on a bottom surface 215 of spine 214. Spine 214 and orthogonal rib 217 may have suitable light transmission properties (i.e., transparency or translucency). As such, indicia or coloration will be magnified by the cylindrical lens formed from the lumen contents such that a magnified image or coloration will be seen by the user through viewport 240. Thus, for example, a thin, colored line provided on surface 215 will appear wider to the user and may appear to occupy the entire width of viewport 240. Sidewalls 244 further enhance viewing by providing a sharp demarcation to the enlarged image. Owing to these features, a clear liquid occupying the lumen would be seen to be perceived as having the color of the coloration provided on a thin line on the spine surface 215. This aspect provides advantages relating to user viewing of syringe gradations, for example, when provided on surface 215. This aspect also provides advantages relating to product branding (i.e., color is a distinguishing feature for many medical products, such as injectable skin treatments and botulinum toxins. Thus, a manufacture desiring to have its otherwise clear liquid product appear in a certain color in a prefilled syringe cartridge may provide coloration on the surface 215 of spine 214 during the manufacturing process and the prefilled (and actually clear, transparent or translucent) liquid in the syringe cartridge would be perceived by the user to have that same color when viewing the contents through viewport 240.

Viewing enhancement features are also provided for side viewing of syringe contents. As best seen in FIGS. 2 and 6, the syringe cartridge includes left side and right side viewing areas 218L and 218R in which the wall thickness surrounding the lumen 202 is sufficiently thin to permit clear viewing of the lumen contents. Moreover, the external surfaces of the lumen wall may be provided with indicia, such as gradations indicating volume, and/or coloration. Thus, the lumen contents can be clearly viewed from either side. And when the lumen is occupied by clear or translucent contents, the indica or coloration provided on the lumen walls in these areas will appear magnified when viewed from the opposite side of the syringe cartridge. In this manner, further information about the syringe contents can be discerned easily by the user.

A syringe plunger 300 includes a plunger button 310, elongate plunger stem 320 and a plunger piston end 330 which may include a plunger piston (not shown) formed of an elastomeric material with one or more sealing rings for sealing and sliding engagement with the interior surface of lumen 202. Plunger 300 may include an enlarged portion 350 which fits within the complementarily shaped receptacle 250 on the syringe cartridge when the plunger is positioned at sufficient depth within the lumen. In a prefilled condition, the syringe cartridge 200 may be provided with the plunger 300 installed such that enlarged portion 350 extends just partially into the receptacle 250 such that enlarged portion 350 cooperates with receptacle 250 throughout the travel of the plunger 300. This arrangement provides increased stability and reduces the risk of the plunger stem bending or buckling during operation.

Referring to FIGS. 2-4, dispensing end 220 includes a U-shaped surface 222 extending around the needle hub receptacle and which is aligned with the bottom surface of spine 214. The lower surface 222 on the dispensing end provides an additional (to surface of spine 214) gripping surface for the user's fingers when the syringe assembly is held and operated. Syringe cartridge 200 includes an enlarged cylindrical lumen portion 250 extending and tapering to a narrow portion of lumen 202. Enlarged lumen portion 250 is adapted to receive a complementarily shaped portion 350 of the syringe plunger to enhance stability and operation of the plunger as it is moved (inserted) relative to the syringe cartridge 200.

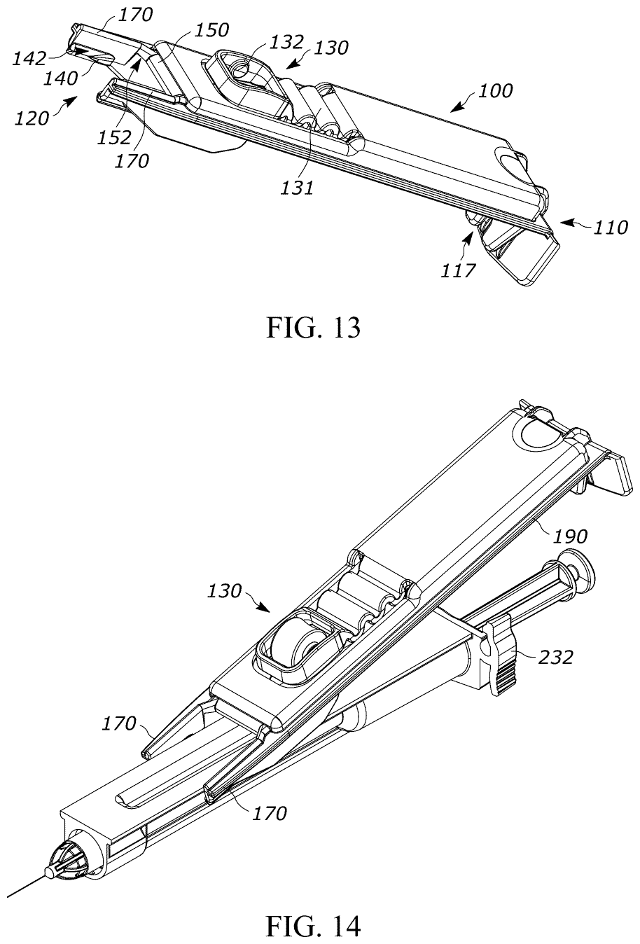

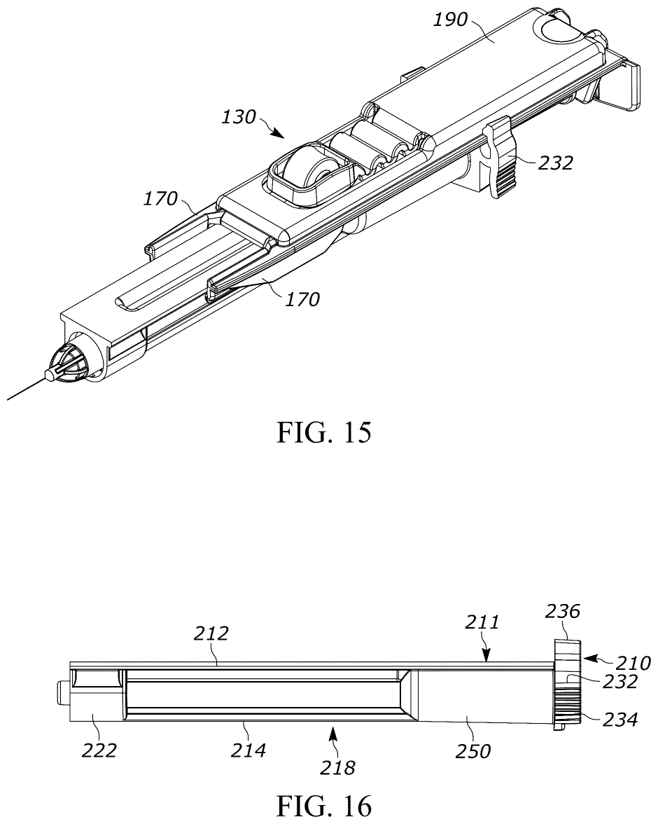

Referring additionally now to FIGS. 8-13, in accordance with aspects of the disclosure, carriage 100 is provided with features for releasable mounting on syringe cartridge 200 and may thus be installed, removed, and reused with multiple ones of the syringe cartridges 200. Carriage 100 includes a syringe button end 110 and a guide end 120. Syringe button end 110 includes a pair of walls 112 and 114 extending from the main body of the carriage 100 and defining a space therebetween for securing the plunger button 310. Wall 114 may include a slot or recess 117 (FIG. 13) for receiving and fastening a narrow portion 322 (FIG. 2) of the syringe plunger 300. As previously described, carriage 100 is configured on guide end 120 to engage and attach to respective sides 212 of the slide 208 when the carriage is installed on the syringe cartridge 200.

Carriage 100 also includes a cantilevered wheel mount 130, which may include a serpentine molded portion 131 for facilitating flexibility and providing a tactile sensation to the user to assist in placement/positioning of the user's thumb on the carriage wheel 180. Carriage wheel or roller 180 is rotatably secured in a receptacle formed as part of the cantilevered wheel mount and having opposing small axles 132 for rotatably securing the roller 180 to the wheel mount. Wheel mount 130 includes a flexible cantilever 136. Thus, the wheel mount 130 may be displaced downward under force from a user's thumb such that cantilever 135 is elastically deformed and roller 180 engages the surface of slide 208. As will be recognized, carriage wheel 180 is shaped and adapted to frictionally engage the flat surface of slide 208 and, when rotated by the user, causes the carriage 100 to move relative to the syringe cartridge 200 in a precise manner. The flexibility of the cantilevered wheel mount 130 allows the user to selectively cause the wheel 180 to engage or disengage the slide surface 210. Roller 180 may be provided with a rubber or elastomeric outer surface for improving traction and frictional engagement with the surface 211 of slide 208.

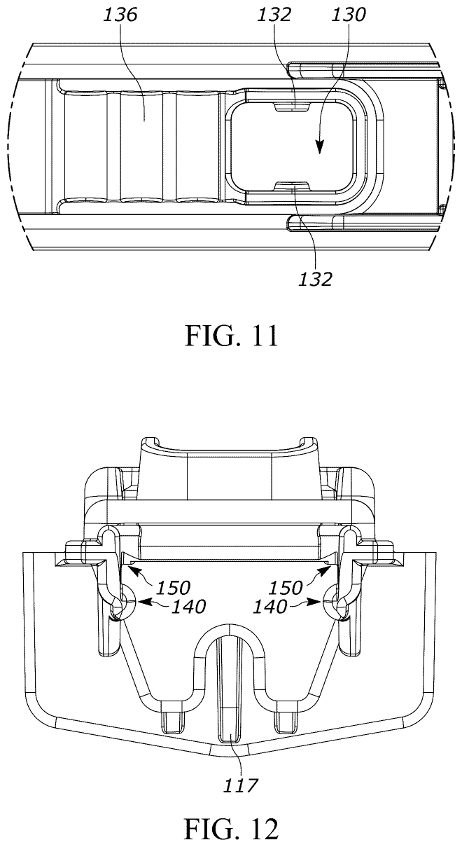

Carriage 100 includes a pair of arms 170 extending in a direction away from the plunger end 110 and defining a viewing area or window 172 which enables a user to view, and draws the user's attention to, an area of the viewport 240 on the syringe cartridge 200 when the carriage is installed. Preferably, the window 172 is positioned such that it frames in the interface between the plunger piston end 330 (FIG. 2) of plunger 300 and the syringe contents. Carriage 100 includes guiding surfaces for capturing and slidably securing the guide end 120 of carriage 100 to the slide 208 of the syringe cartridge 200. Carriage 100 includes a pair of lower tabs or projections 140 (FIG. 12) on arms 170 and having lower guide surfaces 142 for engaging the underside of the outer edges of slide 208. Carriage 100 also includes an upper guide 150 extending laterally and having an upper guide surface 152 to engage the top surface of slide 208. Projections 140 may be dimensioned and spaced to provide for a snap fit of the guide end 120 onto the slide 208 when the carriage 100 is installed on the cartridge 200.

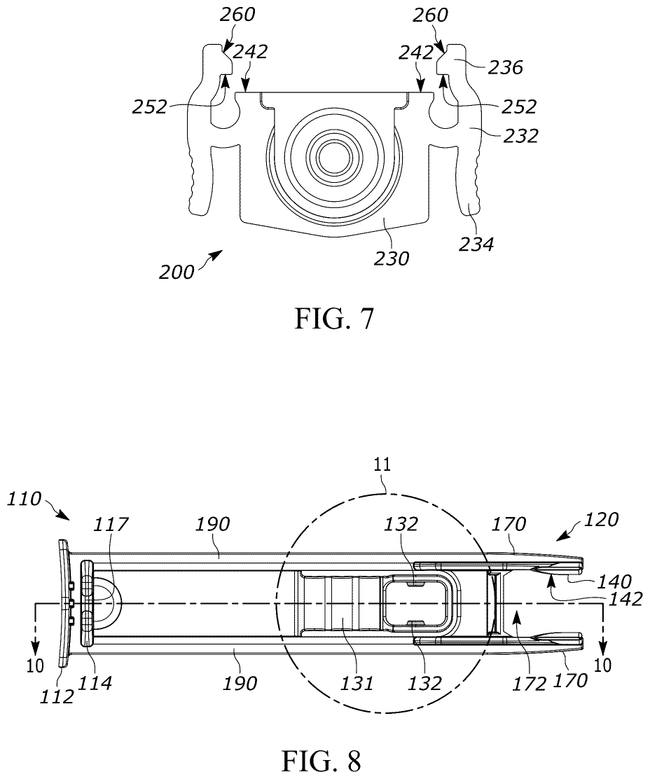

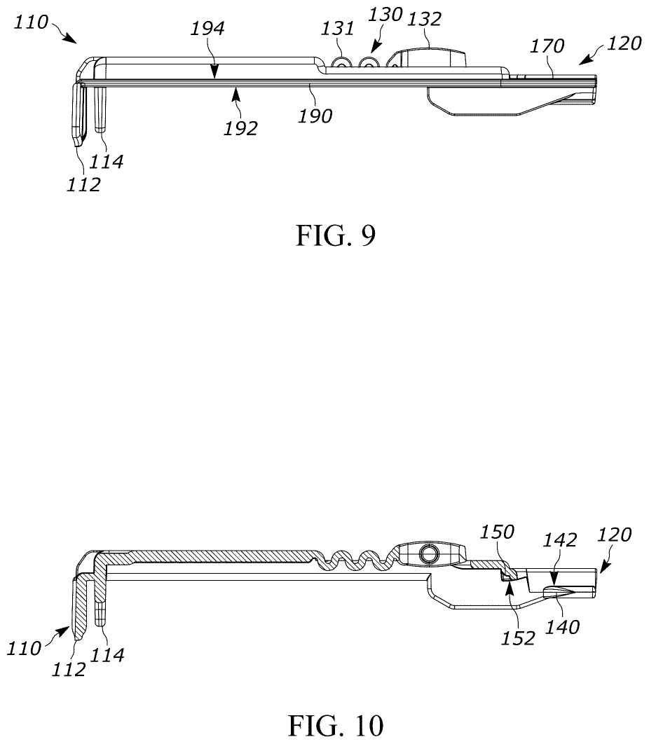

The plunger end 110 of carriage 100 and the syringe cartridge 200 also include features for guiding the carriage 100 for movement relative to the syringe cartridge. Referring to FIG. 7, the syringe cartridge 200 fastening levers 232 include upper guide surfaces 252 arranged to engage top surfaces 194 (FIG. 9) of the outer rails 190 of carriage 100. Syringe cartridge 200 also includes lower guide surfaces 242 which are arranged to engage bottom surfaces 192 of outer rails 190 of carriage 100. A user may install the carriage 100 on the syringe cartridge 200 by first positioning the carriage 100 at an angle to the syringe cartridge 200 (FIG. 14) and positioning the guide end 120 of the carriage 100 in front of the dispensing end 220 of the syringe cartridge. The user then positions the forward end of slide 208 between the lower guide surfaces 140 and the upper guide surface 150 of the carriage 100 and rotates the carriage 100 towards the syringe cartridge 200 such that the plunger end 110 of carriage 100 moves towards the plunger button 310 and the plunger button will be positioned between the walls 112, 114 (with the plunger being installed in the syringe cartridge lumen). The carriage 100 is then “clicked” or snapped into position as the carriage rails 190 are moved downward, engaging the ramped surfaces 260 of the fastening levers 232, moving them outward, and then causing them to snap back to a retaining position in which surfaces 252 and 240 secure the rails 190 for sliding movement relative to the carriage 100. The carriage is then positioned on the cartridge 200 and the user may precisely dispense syringe contents by gripping the syringe assembly such that the user's fingers engage the spine 260 or surfaces 230 on dispensing end or the surface on enlarged portion 250. The user's thumb may be positioned to engage, press on and rotate the wheel 180 to move the carriage 100 and the captured plunger 300 relative to the syringe cartridge 200, thereby dispensing contents.

Release of the carriage from the cartridge 200 after use may be performed by squeezing the fastening arm actuating ends and pivoting the fastening levers 232 such that the rails 190 of the carriage 100 are free to move upward past the surfaces 252. The plunger end 110 of the carriage 100 is pivoted upward such that the plunger button 310 is disengaged. The user then moves the carriage 100 forward relative to the cartridge 200 and disengages the guide end 120 from the slide 208, thereby allowing the carriage 100 to be removed entirely from the syringe cartridge 200.

The syringe cartridge may be provided with an internal lumen diameter that provides a total syringe volume, when the plunger/piston is at the full extent of its travel, of about 0.50 ml, with 50 gradations indicated on the syringe barrel, or in another configuration, about 0.25 ml, with 25 gradations indicated on the syringe barrel. With a syringe length of 4 inches with a 0.50 ml volume and 50 gradations, for example, each incremental 0.01 ml dose may require a controlled a plunger movement on the order of 0.08 inches.

As will be appreciated by those of ordinary skill, the features of the syringe assembly described above facilitates improved visual characteristics as well as small, precise and controlled movements of the syringe plunger for such small incremental doses. Moreover, the snap-on features of the carriage and cartridge have advantages in mass production for pre-filled syringe cartridges, for example, and the reusability of the syringe carriage with multiple syringe cartridges and less waste than completely disposable syringe arrangements.

Although the present implementations have been described with reference to specific example embodiments, it will be evident that various modifications and changes may be made to these embodiments without departing from the broader spirit and scope of the invention as set forth in the claims. Accordingly, the specification and drawings are to be regarded in an illustrative rather than a restrictive sense.

What is claimed is:

1. A syringe cartridge comprising:
a main body including a lumen defined on an interior thereof;
a viewport defined in main body and arranged to permit a user to view syringe contents in the lumen.

2. The syringe cartridge of claim 1, further comprising a spine extending axially along the main body and having a spine surface, wherein the spine surface is arranged such that indicia on the spine surface appear magnified to a user when viewed through contents in the syringe lumen and the viewport.

3. The syringe cartridge of claim 1, further comprising a fastening assembly on the main body, the fastening assembly configured to secure a carriage to the cartridge.

4. The syringe cartridge of claim 1, further comprising a plunger and a carriage adapted to engage an end of the plunger, the carriage configured to slide relative to the main body.

5. The syringe cartridge of claim 4, further comprising a roller on the carriage adapted to frictionally engage the syringe cartridge.

6. The syringe cartridge of claim 5, further comprising a flexible mount on the carriage for mounting the roller.

7. The syringe cartridge of claim 1, wherein the main body has a wall thickness surrounding the lumen and wherein the viewport extends to a depth such that the syringe contents are viewed through a thickness of material that is less than or equal to the wall thickness surrounding the lumen.

8. The syringe cartridge of claim 1, further comprising a lateral viewing area defined on the main body and adapted to permit a user to view the syringe contents from a side of the syringe body.

9. The syringe cartridge of claim 1, wherein the viewport is an axially extending trough formed in the syringe cartridge.

10. The syringe of claim 9, wherein the trough extends to a depth in the main body such that syringe contents are clearly visible to a user.

11. The syringe of claim 3, wherein the fastening assembly comprises a guide surface on the syringe cartridge main body for slidably engaging a carriage and at least one releasable fastener for securing the carriage to the syringe cartridge.

12. A syringe cartridge comprising:
a main body including a lumen defined on an interior thereof;
a slide extending from the main body and including a generally planar surface;
a carriage fastening assembly disposed on the main body, the carriage fastening assembly configured to releasably and slidably secure a carriage to the syringe cartridge.

13. The syringe cartridge of claim 12, further comprising a syringe carriage adapted to engage a syringe plunger and adapted to slide relative to the syringe cartridge to move the plunger relative thereto, the syringe carriage having at least one rail defined thereon, wherein the fastening assembly is configured to engage and retain the at least one rail.

14. The syringe cartridge of claim 12, further comprising a syringe carriage adapted to engage a syringe plunger and adapted to slide relative to the syringe cartridge to move the plunger relative thereto, wherein the carriage fastening assembly includes a pair of pivoting levers configured to capture and secure a carriage for sliding movement relative to the syringe cartridge.

15. The syringe cartridge of claim 12, further comprising a syringe carriage adapted to engage a syringe plunger and adapted to slide relative to the syringe cartridge to move the plunger relative thereto, wherein the syringe carriage includes a pair of projections configured to secure the carriage to the slide with a snap fit and to guide the carriage for sliding movement on the slide.

16. The syringe cartridge of claim 12, further comprising a syringe carriage adapted to engage a syringe plunger and adapted to slide relative to the syringe cartridge to move the plunger relative thereto, wherein the syringe carriage includes a roller configured to frictionally engage the slide and allow a user to precisely move the carriage relative to the syringe cartridge.

17. The syringe cartridge of claim 12, wherein the fastening assembly comprises a pair of lower guide surfaces arranged to slidingly engage a lower surface of the carriage, and pair of upper guide surface arranged to slidingly engage an upper surface of the carriage such that the carriage is captured for sliding movement between the lower guide surfaces and upper guide surfaces.
